(19) United States
(12) Patent Application Publication
(10) Pub. No.: US 2019/0136173 A1
(43) Pub. Date: May 9, 2019

(54) PERFUSION APPARATUS FOR USE IN BIOREACTOR SYSTEMS

(71) Applicant: Lonza Ltd., Visp (CH)

(72) Inventors: Yonatan Levinson, Silver Spring, MD (US); Eytan Abraham, Potomac, MD (US); Siddharth Gupta, Seattle, WA (US)

(21) Appl. No.: 16/146,030

(22) Filed: Sep. 28, 2018

Related U.S. Application Data
(60) Provisional application No. 62/565,187, filed on Sep. 29, 2017.

(51) Int. Cl. C12M 1/00; C12M 1/12; C12M 1/06

(52) U.S. Cl. CPC C12M 29/10; C12M 23/06; C12M 27/02; C12M 23/28; C12M 25/14; C12M 25/02

(57) ABSTRACT

A perfusion apparatus as disclosed for withdrawing a fluid medium from a bioreactor during the growth of a cell culture on microcarriers within the bioreactor. Also disclosed is a method for culturing cells in a bioreactor contained on microcarriers. The perfusion apparatus includes a hollow tubular member attached to a filter member. The filter member has a pore size and volume capable of withdrawing a fluid medium at a relatively high flow rate from the bioreactor.

### RELATED APPLICATIONS

The present application is based on and claims priority to U.S. Provisional Patent Application Ser. No. 62/565,187 having a filing date of Sep. 29, 2017, which is incorporated herein by reference in its entirety.

### BACKGROUND

Bioreactors, which are apparatuses in which biological reactions or processes can be carried out on a laboratory or industrial scale, are used widely within the biopharmaceutical industry. Bioreactors can be used in batch applications, where biological materials supplied to a bioreactor remain in the bioreactor until the end of the reaction time. Alternatively, bioreactors can be used in perfusion applications, wherein the fluid medium contained within the bioreactor is periodically or continuously removed and resupplied to the bioreactor in order to replenish nutrients contained within the fluid medium and for possibly removing damaging by-products that are produced during the process.

In some bioreactor systems, microcarriers are added to the bioreactor to promote cell growth. For instance, cells can adhere to the surface of the microcarriers for further growth and propagation. In this manner, the microcarriers can provide greater surface area for cell culture growth within the reactor. In fact, some anchorage-dependent cells, such as certain animal cells, need to attach to a surface in order to grow and divide.

Microcarriers can be made from various different materials, including polymers. The microcarriers can have any suitable shape and, in some applications, comprise round beads. The microcarriers can generally have a particle size of from about 200 microns to about 350 microns. In some systems, the microcarriers are suspended within a culture medium caused by general agitation which optimizes and maximizes the growing conditions within the bioreactor system.

One problem experienced in the past in bioreactor systems containing microcarriers is the ability to rapidly remove fluids from the bioreactor without also removing the microcarriers or damaging the microcarriers or the cells growing on the microcarriers. Attempts to increase flow rates out of bioreactor systems containing microcarriers can result in impeded or obstructed flow due to inappropriately sized dip tubes, clogging caused by the microcarriers, and/or collapsed tubing due to the suction head created upstream from a pump driving the flow. In view of the above, a need exists for an apparatus and method for rapidly removing fluid culture media from a bioreactor containing microcarriers.

### SUMMARY

In general, the present disclosure is directed to a perfusion apparatus capable of removing a fluid culture medium from bioreactors at relatively high flow rates without damaging the bioreactor or cells being grown within the reactor. More particularly, the present disclosure is directed to a perfusion apparatus that is particularly designed to remove culture fluid mediums from bioreactors at relatively high flow rates that contain microcarriers. As will be described in greater detail below, the perfusion apparatus is particularly well adapted for removing fluids without removing or harming the microcarriers or cells attached to the microcarriers. The present disclosure is also directed to a method for promoting cell growth in a bioreactor system in which the perfusion apparatus is used to remove culture fluid medium for replenishment and further growth of the cells.

In one embodiment, for instance, the present disclosure is directed to a perfusion apparatus that includes a hollow tubular member for perfusing fluid from a bioreactor. The hollow tubular member may have a length sufficient for insertion into a bioreactor. For instance, the hollow tubular member can have a length sufficient to extend towards the bottom of a bioreactor. The hollow tubular member may extend through a port in the top or side of the bioreactor. The hollow tubular member has a first end defining a first opening and a second and opposite end defining a second opening. The second opening is for insertion into a fluid medium in a bioreactor and for withdrawing the fluid medium. The second opening of the hollow tubular member can have a cross-sectional area designed to be capable of withdrawing a desired volumetric flow rate from the bioreactor.

In accordance with the present disclosure, the perfusion apparatus further includes a filter member located and attached to the second end of the hollow tubular member. The filter member completely surrounds and encloses the second opening. The filter member has a length that extends past the second end of the hollow tubular member and defines an enclosed volume. The enclosed volume is of a size sufficient for a desired fluid flow rate even when the bioreactor contains microcarriers. For example, in one embodiment, the ratio between the cross-sectional area of the second opening and the surface area of the filter member is from about 1:5 to about 1:200, such as from about 1:15 to about 1:100. As used herein, the surface area of the filter member is the total surface area of the porous portion of the filter member that allows fluids to enter the hollow tubular member.

In one embodiment, the filter member comprises a porous mesh. The porous mesh, for instance, may comprise a screen, such as a stainless steel screen or polymer mesh. In one embodiment, the mesh has an average pore size of greater than about 60 microns, such as greater than about 70 microns, such as greater than about 80 microns. For instance, the average pore size of the mesh can be from about 60 microns to about 150 microns. In one embodiment, the pores on the mesh all have a generally uniform size. In one embodiment, the pore size of the mesh is from about 60 microns to about 150 microns.

The hollow tubular member and the second opening can generally have a diameter of greater than about 2 mm, such as greater than about 4 mm, such as greater than about 8 mm, such as greater than about 10 mm, such as greater than about 12 mm, such as greater than about 14 mm, such as greater than about 16 mm, such as greater than about 18 mm, such as greater than about 20 mm. The diameter of the hollow tubular member is generally less than about 50 mm, such as less than about 30 mm, such as less than about 20 mm, such as less than about 14 mm. The hollow tubular member can be made from various different materials, such as stainless steel or a polymer. In one embodiment, the hollow tubular member is straight from the first end to the second end. In an alternative embodiment, the hollow tubular structure can have a shape such that the second end does not interfere with an impeller that can be rotating in the bioreactor. For example, in one embodiment, the hollow tubular member can include a first straight section, a second straight section, and an angular section positioned between the first straight section and the second straight second. The angled section can extend from the first straight section at an angle of from about 25° to about 45°. Similarly, the angled section can extend from the second straight section at an angle of from about 25° to about 45°. In one embodiment, the first straight section and the second straight section are parallel to a vertical axis that extends through the bioreactor.

In one embodiment, the hollow tubular member can also include an angular member located at the second end. The hollow tubular member can include a straight member that transitions into the angular member. The angular member can be at an angle to the straight section of from about 50⁰ to about 90°. For example, in one embodiment, the angular member forms a right angle at the end of the hollow tubular member. In this regard, when the perfusion apparatus is extended into a bioreactor, the angular member can be positioned towards the bottom of the bioreactor and can be generally parallel with the bottom surface of the bioreactor. For instance, in one embodiment, the angular member can be designed to place the filter member below an impeller contained within the bioreactor.

In one embodiment, the hollow tubular member and the filter member can be completely enclosed for sterile closed connection to a port of the bioreactor. A plastic, flexible bellows can enclose the hollow tubular member and the filter member. A sterile connection port may be attached to one end of the bellows. The bioreactor port may have a matching sterile connector. When the matching sterile connectors of the bioreactor and the bellows are connected, the bellows may be collapsed and the filter member and hollow tubular member may be inserted into the bioreactor port.

In one embodiment, the perfusion apparatus can include a filter member on a side or bottom wall of the bioreactor. The filter member can be a mesh patch on the side or bottom wall of the bioreactor. A flexible cone can connect the mesh patch to a hollow tubular member for output of fluid from the bioreactor.

The present disclosure is also directed to a method for growing a cell culture within a bioreactor. The method includes inoculating cells within a bioreactor containing a microcarrier. The biological cells can attach to the microcarrier for continued cell growth. The bioreactor can contain a fluid medium for providing nutrients and food to the growing cell culture.

In accordance with the present disclosure, the method further includes the step of continuously or periodically removing the fluid medium from the bioreactor using the perfusion apparatus as described above. In one embodiment, for instance, the perfusion apparatus can be designed to remove the fluid medium at a rate of greater than about 20 L per day, such as greater than about 25 L per day, such as greater than about 30 L per day, such as greater than about 35 L per day, such as greater than about 40 L per day, such as greater than about 45 L per day, such as greater than about 50 L per day.

As the fluid medium is removed from the bioreactor, a new fluid medium is added to the bioreactor for further promoting growth of the biological cells attached to the microcarriers. After a desired amount of cell growth, a release agent can be added to the bioreactor causing the cells to separate from the microcarriers. The cells can then be harvested and used as desired.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 is a cross-sectional view of one embodiment of a bioreactor system in accordance with the present disclosure;

FIG. 2 is a side view of one embodiment of a perfusion apparatus made in accordance with the present disclosure;

FIG. 3 is a side view of another embodiment of a perfusion apparatus made in accordance with the present disclosure;

FIG. 4A is a perspective view of one embodiment of a filter member attached to a perfusion apparatus in accordance with the present disclosure;

FIG. 4B is a side view of the filter member illustrated in FIG. 4A;

FIG. 5 is a side view of another embodiment of a perfusion apparatus made in accordance with the present disclosure;

FIG. 6 is a perspective view of another embodiment of a bioreactor system in accordance with the present disclosure;

FIGS. 7A through 7C are perspective views of one embodiment of bioreactor system having a sterile closed connection between the bioreactor and the perfusion apparatus;

FIG. 8A is a perspective view of another embodiment of a bioreactor system in accordance with the present disclosure;

FIG. 8B is a side view of a perfusion apparatus that may be used with the bioreactor system illustrated in FIG. 8A;

FIG. 9 is a side view of another embodiment of a bioreactor system in accordance with the present disclosure;

FIGS. 10A and 10B illustrate data resulting from tests of the accuracy of a perfusion apparatus made in accordance with the present invention.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to methods and systems for cultivating and propagating cells and/or cell products in a bioreactor. According to the present disclosure, biological cells, such as mammalian cells, are combined with a suitable microcarrier within a bioreactor. The cells attach to the microcarrier for promoting cell growth. The bioreactor contains a fluid medium, such as a fluid growth medium. The biological cells are cultivated under suitable conditions and in a suitable culture medium for promoting cell reproduction and growth until a desired amount of cells can be harvested from the bioreactor.

In accordance with the present disclosure, in addition to containing one or more microcarriers, the bioreactor is designed to be run in the perfusion mode during cell culturing processes. In particular, the fluid medium contained within the bioreactor is continuously or at least periodically removed and replenished. In the past, problems have been experienced in removing liquid mediums from bioreactors containing microcarriers at flow rates sufficient to maintain optimum growth conditions within the reactor. In this regard, the present disclosure is directed to a perfusion apparatus that is capable of rapidly removing fluid medium from the bioreactor without also removing the microcarriers, without damaging the microcarriers, and/or without damaging the cell culture within the bioreactor.

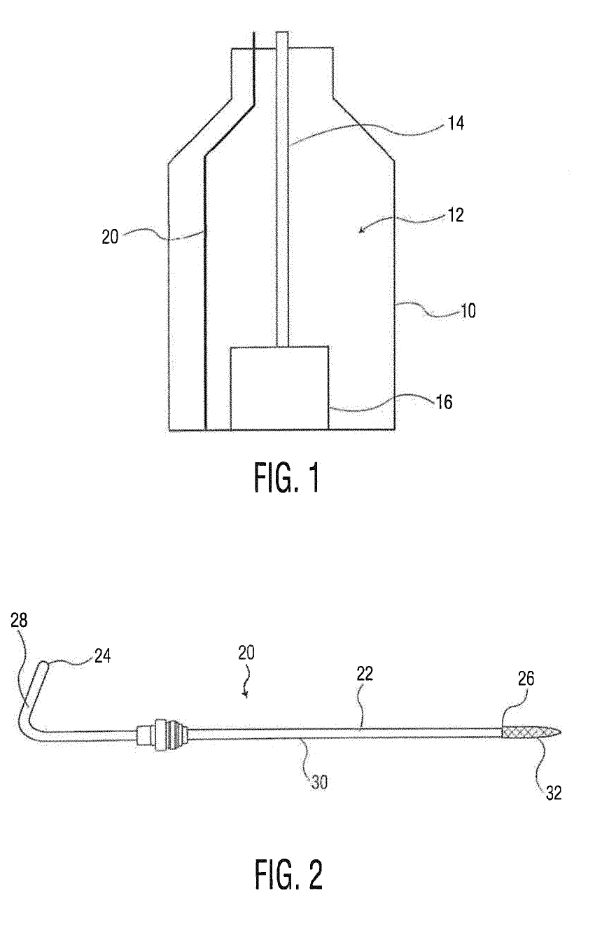

Referring to FIG. 1, one embodiment of a bioreactor system in accordance with the present disclosure is shown. The bioreactor system includes a bioreactor 10. The bioreactor 10 comprises a hollow vessel or container that includes a bioreactor volume 12 for receiving a cell culture attached to microcarriers suspended within a fluid growth medium. As shown in FIG. 1, the bioreactor system can further include a rotatable shaft 14 coupled to an agitator such as an impeller 16.

The bioreactor 10 can be made from various different materials. In one embodiment, for instance, the bioreactor 10 can be made from metal, such as stainless steel. Metal bioreactors are typically designed to be reused.

Alternatively, the bioreactor 10 may comprise a single use bioreactor made from a flexible polymer film. The film or shape conforming material can be liquid impermeable and can have an interior hydrophilic surface. In one embodiment, the bioreactor 10 can be made from a flexible polymer film that is designed to be inserted into a rigid structure, such as a metal container for assuming a desired shape. Polymers that may be used to make the flexible polymer film include polyolefin polymers, such as polypropylene and polyethylene. Alternatively, the flexible polymer film can be made from a polyamide. In still another embodiment, the flexible polymer film can be formed from multiple layers of different polymer materials. In one embodiment, the flexible polymer film can be gamma irradiated.

The bioreactor 10 can have any suitable volume. For instance, the volume of the bioreactor 10 can be from 100 mL to about 10,000 L or larger. For example, the volume 12 of the bioreactor 10 can be greater than about 0.5 L, such as greater than about 1 L, such as greater than about 2 L, such as greater than about 3 L, such as greater than about 4 L, such as greater than about 5 L, such as greater than about 6 L, such as greater than about 7 L, such as greater than about 8 L, such as greater than about 10 L, such as greater than about 12 L, such as greater than about 15 L, such as greater than about 20 L, such as greater than about 25 L, such as greater than about 30 L, such as greater than about 35 L, such as greater than about 40 L, such as greater than about 45 L. The volume of the bioreactor 10 is generally less than about 20,000 L, such as less than about 15,000 L, such as less than about 10,000 L, such as less than about 5,000 L, such as less than about 1,000 L, such as less than about 800 L, such as less than about 600 L, such as less than about 400 L, such as less than about 200 L, such as less than about 100 L, such as less than about 50 L, such as less than about 40 L, such as less than about 30 L, such as less than about 20 L, such as less than about 10 L. In one embodiment, for instance, the volume of the bioreactor can be from about 1 L to about 5 L. In an alternative embodiment, the volume of the bioreactor can be from about 25 L to about 75 L. In still another embodiment, the volume of the bioreactor can be from about 1,000 L to about 5,000 L.

In addition to the impeller 16, the bioreactor 10 can include various additional equipment, such as baffles, spargers, gas supplies, ports, and the like which allow for the cultivation and propagation of biological cells. In addition, the bioreactor system can include various probes for measuring and monitoring pressure, foam, pH, dissolved oxygen, dissolved carbon dioxide, and the like.

In one embodiment, the bioreactor 10 includes a top that defines a plurality of ports. The ports can allow supply lines and feed lines into and out of the bioreactor 12 for adding and removing fluids and other materials. In addition, the bioreactor system can be placed in association with a load cell for measuring the mass of the culture within the bioreactor 10.

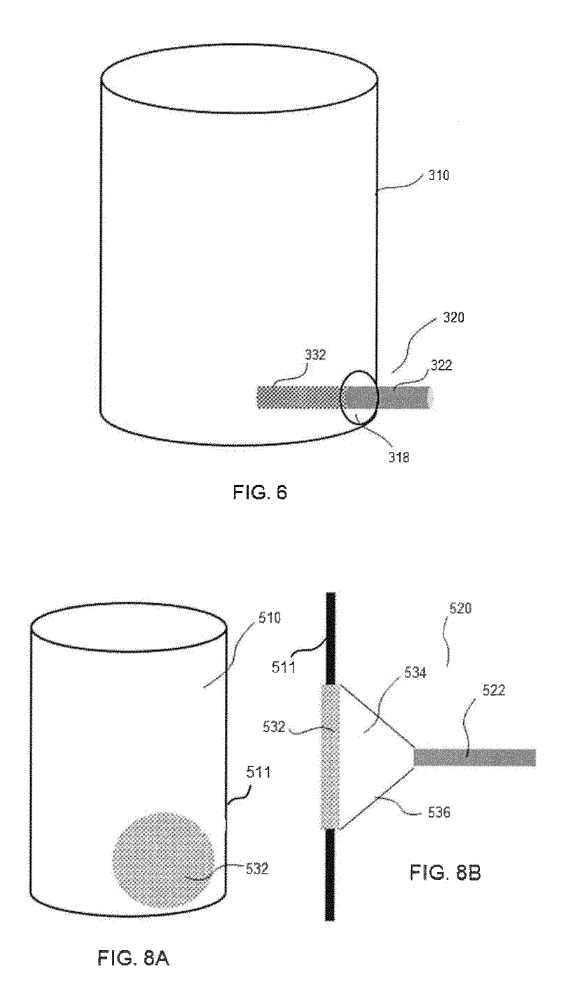

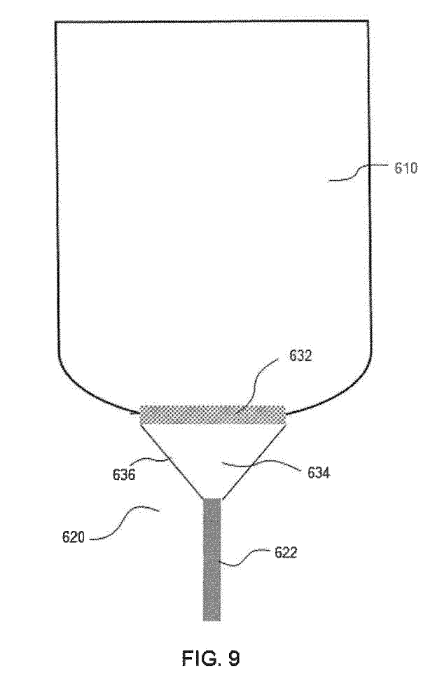

In an alternative embodiment, the plurality of ports can be located at different locations on the bioreactor 10. For instance, in one embodiment, the ports can be located on a side wall of the bioreactor, as shown in FIGS. 6-8. In another embodiment, the ports can be located at the bottom of the bioreactor, as shown in FIG. 9. For example, a bioreactor made from a flexible polymer film may include ports located on the bottom of the vessel.

As shown in FIG. 1, the bioreactor 10 can include a rotatable shaft 14 attached to at least one impeller 16. The rotatable shaft 14 can be coupled to a motor for rotating the shaft 14 and the impeller 16. The impeller 16 can be made from any suitable material, such as a metal or a biocompatible polymer. Examples of impellers suitable for use in the bioreactor system include hydrofoil impellers, high-solidity pitch-blade impellers, high-solidity hydrofoil impellers, Rushton impellers, pitched-blade impellers, gentle marine-blade impellers, and the like. In addition, the rotatable shaft 14 can be coupled to a single impeller 16 as shown in FIG. 1 or can be coupled to two or more impellers. When containing two or more impellers, the impellers can be spaced apart along the rotating shaft 14. In one embodiment, the impeller 16 is rotated an amount sufficient to maintain microcarriers contained in the bioreactor 10 in suspension in a fluid medium without damaging biological cells that are attached to the microcarriers.

In one embodiment, the bioreactor system can also include a controller which may comprise one or more programmable devices or microprocessors. The controller can be used to maintain optimum conditions within the bioreactor 10 for promoting cell growth. The controller, for instance, can be in communication and control thermal circulators, load cells, control pumps, and receive information from various sensors and probes. For instance, the controller may control and/or monitor the pH, dissolved oxygen tension, dissolved carbon dioxide, the temperature, the agitation conditions, alkali condition, fluid growth medium condition, pressure, foam levels, and the like. For example, based upon pH readings, the controller may be configured to regulate pH levels by adding requisite amounts of acid or alkali. The controller may also use a carbon dioxide gas supply to decrease pH. Similarity, the controller can receive temperature information and control fluids being fed to a water jacket surrounding the bioreactor for increasing or decreasing temperature.

In accordance with the present disclosure, the bioreactor 10 can also be in communication with a perfusion apparatus 20 as shown in FIG. 1. The perfusion apparatus 20 can extend through a port within the top of the bioreactor 10. As shown, the perfusion apparatus 20 can extend into the bioreactor 10 and be placed adjacent to the bottom of the bioreactor without interfering with the impeller 16. The perfusion apparatus 20 is for continuously or periodically withdrawing liquid medium from the bioreactor 10 without withdrawing microcarriers contained within the bioreactor. The perfusion apparatus 20 of the present disclosure, for instance, can withdraw fluid at a relatively high flow rate without also removing the microcarriers or damaging cells attached to the microcarriers. The microcarriers, for instance, can comprise beads or small particles that are biologically compatible and provide an attachment site for propagating biological cells. In one embodiment, for instance, the microcarriers can be made from a polymer, such as a polysaccharide. In one particular embodiment, for instance, the microcarriers can be made from dextran. The microcarriers can have a particle size or diameter of from about 150 microns to about 400 microns.

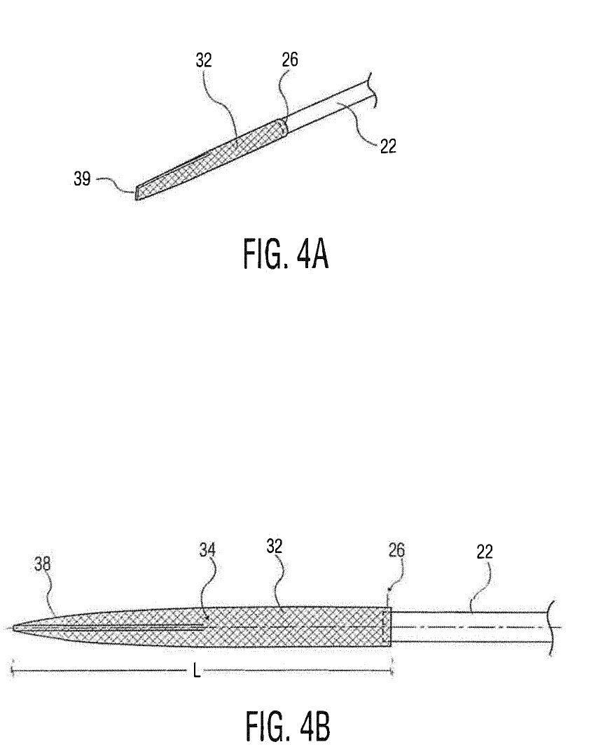

Referring to FIGS. 2, 4A and 4B, one embodiment of a perfusion apparatus 20 that may be used in accordance with the present disclosure is shown. Referring to FIG. 2, the perfusion apparatus 20 includes a hollow tubular member 22. The hollow tubular member 22 can include a first end 24 that defines a first opening and a second and opposite end 26 that defines a second opening. The hollow tubular member 22 can be made from any suitable material that is biologically compatible with cell cultures. For example, the hollow tubular member 22 can be made from a metal, such as stainless steel.

In an alternative embodiment, the hollow tubular member can be made from a polymer. In one embodiment, for instance, the perfusion apparatus 20 can be designed to be discarded after a single use. In this embodiment, the hollow tubular member 22 can be made from a polymer material. For instance, the hollow tubular member can be made from a polyolefin, such as polypropylene or polyethylene. Alternatively, the hollow tubular member 22 can be made from a polyamide. Otherwise, the hollow tubular member 22 can be made from a plastic material that can be gamma irradiated.

The hollow tubular member 22 can be flexible or rigid. The hollow tubular member 22, the first opening, and the second opening can generally have a diameter sized for the particular application and the amount of fluid needed to be withdrawn from the bioreactor 10. For instance, the diameter of the hollow tubular member 22 can generally be greater than about 2 mm, such as greater than about 4 mm, such as greater than about 6 mm, such as greater than about 8 mm, such as greater than about 10 mm. The diameter of the hollow tubular member 22 is generally less than about 40 mm, such as less than about 30 mm, such as less than about 20 mm, such as less than about 15 mm, such as less than about 11 mm, such as less than about 10 mm, such as less than about 8 mm.

The first end 24 of the hollow tubular member 22 can include a tubing connection for connecting the hollow tubular member 22 to plastic tubing. The tubing connection can be any of various weldable tubing types. The outer diameter of the tubing connection of the first end 24 can generally have an outer diameter sized for the particular application and the amount of fluid needed to be withdrawn from the bioreactor. For instance, the outer diameter of the tubing connection can generally be about 3 mm or more, such as about 6 mm or more, such as about 13 mm or more, such as about 19 mm or more, such as about 26 mm. The outer diameter of the tubing connection is generally about 26 mm or less.

The hollow tubular member 22 can be made from a single piece of material or can be made from multiple pieces connected together. The hollow tubular member 22 can be straight from the first end 24 to the second end 26. Alternatively, the hollow tubular member 22 can include an angular member 28 as shown in FIG. 2. In the embodiment illustrated in FIG. 2, the angular member 28 extends from the bioreactor 10 for directing the flow of fluids out of the bioreactor in a desired direction. The angular member 28 as shown in the figures generally makes a right angle with a straight section 30 of the hollow tubular member 22. The angular member 28, however, can be at any suitable angle with respect to the straight or vertical section 30 of the hollow tubular member 22.

When used to remove fluids from the bioreactor 10, the perfusion apparatus should have a length sufficient such that the second end 26 of the hollow tubular member 22 resides adjacent to the bottom surface of the bioreactor 10. In this regard, the straight section 30 of the perfusion apparatus 20 generally has a length greater than the length (or depth) of the bioreactor 10. For instance, the length of the straight section 30 can be greater than about 110%, such as greater than about 120%, such as greater than about 150% of the length of the bioreactor 10. In general, the straight section 30 is less than about 500%, such as less than 300%, such as less than about 200% of the length of the bioreactor 10.

In accordance with the present disclosure, the perfusion apparatus 20 further includes a filter member 32 positioned at the second end of 26 of the hollow tubular member 22. The filter member 32 is shown in greater detail in FIGS. 4A and 4B. The filter member 32 is sufficiently porous to permit a relatively high flow rate of fluid medium through the perfusion apparatus 20 without permitting the flow of microcarriers or otherwise damaging the microcarriers. For example, in one embodiment, the filter member 32 can be made from a porous mesh, such as a stainless steel screen. Alternatively, the filter member 32 can be made from a polymer material. For instance, in one embodiment, the filter member 32 can be made from a polyamide screen mesh. A polymer mesh, for instance, may be more flexible and less susceptible to damage than a filter element made from a metal. A perfusion apparatus 20 having a polymer hollow tubular member 22 and filter member 32 may further include a polymer shell (not shown) surrounding the filter member 32.

The mesh can have a desired pore size. The pore size can be uniform over the mesh or can be non-uniform. In one embodiment, the mesh has a pore size of greater than about 60 microns, such as greater than about 70 microns, such as greater than about 80 microns, such as greater than about 90 microns. The pore size is generally less than about 150 microns, such as less than about 130 microns, such as less than about 120 microns, such as less than about 110 microns. The above pore sizes have been found to optimize fluid flow in a non-disruptive manner. Smaller pore sizes, for instance, do not permit sufficient flow rates and can experience problems with blockage. In other embodiments, however, smaller pore sizes may be desired. For instance, in other embodiments, the pore sizes can be less than about 50 microns. For example, filter elements made from polymers may have smaller pore sizes. When made from a polymer, for instance, the pore size can be from about 18 microns to about 50 microns, such as from about 20 microns to about 30 microns.

Referring to FIGS. 4A and 4B, the filter member 32 is illustrated in greater detail. As shown, the filter member 32 is attached to the second end 26 of the hollow tubular member 22. For instance, in the embodiment illustrated, the filter member 32 completely surrounds and encloses the opening located at the second end 26 of the hollow tubular member 22. The filter member 32 can be attached to the hollow tubular member 22 using any suitable method or technique. For instance, the filter member 32 can be welded to the hollow tubular member 22, can be adhered to the hollow tubular member 22 or can be mechanically attached to the hollow tubular member. In one particular embodiment, for instance, the filter member 32 can be resin welded to the hollow tubular member 22.

As shown in FIG. 4B, in one embodiment, the filter member 32 has a length L that extends beyond the second end 26 of the hollow tubular member 22. In this manner, the filter member 32 defines an enclosed volume 34. The size of the enclosed volume 34 can depend upon the flow requirements of the system and can be proportional to the cross-sectional area of the opening of the second end 26. For instance, the enclosed volume 34 can be of a size sufficient to allow sufficient fluid flow through the filter member and into the hollow tubular member 22 that may be desired for a particular application. The enclosed volume 34, for instance, increases the surface area of the filter member 32 and thus provides more area for fluids to enter the filter member and allows for greater flow rates through the hollow tubular member 22.

For instance, in one embodiment, the ratio between the cross-sectional area of the opening at the second end 26 to the surface area of the filter member 32 can be greater than about 1:5, such as greater than about 1:10, such as greater than about 1:15, such as greater than about 1:20, such as greater than about 1:25, such as greater than about 1:30, such as greater than about 1:35, such as greater than about 1:40. The ratio between the cross-sectional area of the opening of the second end 26 and the surface area 34 of the filter member 32 can generally be less than about 1:1000, such as less than about 1:500, such as less than about 1:200, such as less than about 1:150, such as less than about 1:100, such as less than about 1:80. For example, when the second opening of the second end 26 has a diameter of from about 2 mm to about 20 mm, the filter member 32 can have a length L of generally greater than about 20 mm, such as greater than about 30 mm, such as greater than about 40 mm, such as greater than about 50 mm, and generally less than about 500 mm, such as less than about 300 mm, such as less than about 100 mm.

In the embodiment illustrated in FIGS. 4A and 4B, the filter member 32 has an elongated shape that terminates at a sloped end 38. It should be understood, however, that the filter member 32 can have any suitable shape. The shape of the filter member 32, for instance, may depend upon a shape that maximizes surface area while being capable of being conveniently placed in the bioreactor 10.

In accordance with the present disclosure, the cross-sectional area of the hollow tubular member 22, the enclosed volume 34 of the filter member 32, and the pore size of the filter member 32 are all selected so as to optimize flow rates. In particular, the perfusion apparatus 20 of the present disclosure is designed to allow for relatively high flow rates out of the bioreactor 10. In one embodiment, for instance, the flow rate through the perfusion apparatus 20 can depend upon the volume of the bioreactor 10. For example, the perfusion apparatus 20 can be designed to withdraw greater than about 50% of the volume of the bioreactor, such as greater than about 60% of the volume of the bioreactor, such as greater than about 70% of the volume of the bioreactor, such as greater than about 80% of the volume of the bioreactor, such as greater than about 90% of the volume of the bioreactor, such as greater than about 100% of the volume of the bioreactor, such as greater than about 110% of the volume of the bioreactor, such as greater than about 120% of the volume of the bioreactor, such as greater than about 130% of the volume of the bioreactor, such as greater than about 140% of the volume of the bioreactor, such as greater than about 150% of the volume of the bioreactor per day (24 hours). In general, the flow rate through the perfusion apparatus 20 is generally less than about 500% of the volume of the bioreactor per day, such as less than about 200% of the bioreactor volume per day.

In one particular example, the perfusion apparatus 20 is designed to withdraw greater than about 20 L of fluid per day, such as greater than about 30 L of fluid per day, such as greater than about 40 L of fluid per day, and generally less than about 100 L per fluid per day out of the bioreactor 10.

The embodiment of the perfusion apparatus 20 as shown in FIG. 2 includes a straight or vertical section 30 that is intended to be inserted into the bioreactor 10. Once inserted in to the bioreactor 10, the straight or vertical section 30 remains substantially parallel with a vertical axis of the bioreactor and/or with the rotatable shaft 14. Thus, the straight or vertical section 30 has a length that is at least as long as the length or depth of the bioreactor 10. In one embodiment, however, the straight or vertical section 30 may interfere with the impeller 16 contained within the bioreactor 10. Thus, in other embodiments, the shape of the perfusion apparatus 20 can be altered for providing a better fit within the bioreactor.

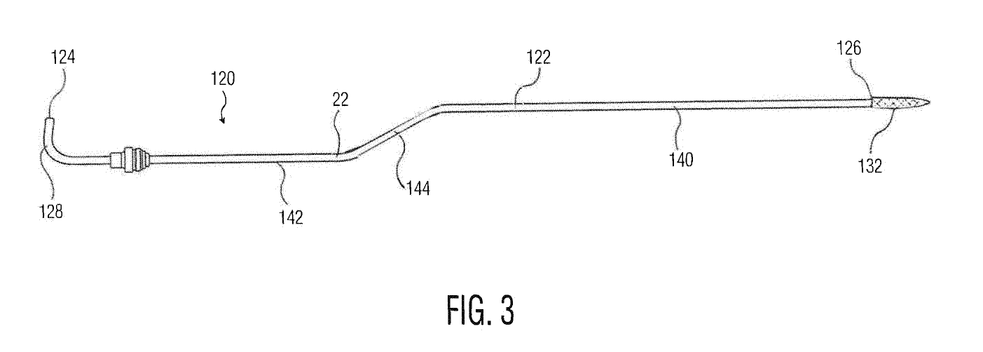

For example, referring to FIG. 3, another embodiment of a perfusion apparatus 120 is shown. The perfusion apparatus 120 includes a hollow tubular member 122 including a first end 124 and a second and opposite end 126. Attached to the second end 126 is a filter member 132 made in accordance with the present disclosure. The hollow tubular member 122 further includes an angular member 128 positioned at the first end 124.

In the embodiment illustrated in FIG. 3, the perfusion apparatus 120 includes a first straight section 140, a second straight section 142, and an angular section 144. The angular section 144 is positioned in between the first straight section 140 and the second straight section 142. As shown in FIG. 1, the angular section 144 can be included in the hollow tubular member 22 in order to prevent the perfusion apparatus 120 from interfering with an impeller 16 contained within the bioreactor 10. In particular, the angular section 144 positions the second end 126 of the hollow tubular member 122 adjacent to the wall of the bioreactor 10. In one embodiment, the angular section 144 can form an angle with the first straight section 140 of from about 10° to about 80°, such as from about 25° to about 45°. For example, the angle between the angular section 144 and the first straight section 140 can generally be greater than about 20°, such as greater than about 30°, such as greater than about 40°, and generally less than about 60°, such as less than about 50°. Similarly, the angle between the angular section 144 and the second straight section 142 can be from about 10° to about 80°, such as from about 25° to about 45°.

The length of the straight sections 140 and 142 and the length of the angular section 144 can also vary depending upon the geometry of the bioreactor 10 and various other factors. In one embodiment, for instance, the angular section 144 can be greater than about 5%, such as greater than about 10%, such as greater than about 15%, such as greater than about 20%, and generally less than about 50%, such as less than about 40%, such as less than about 30%, such as less than about 20%, of the total length of the first straight section 140, the second straight section 142, and the angular section 144 taken together.

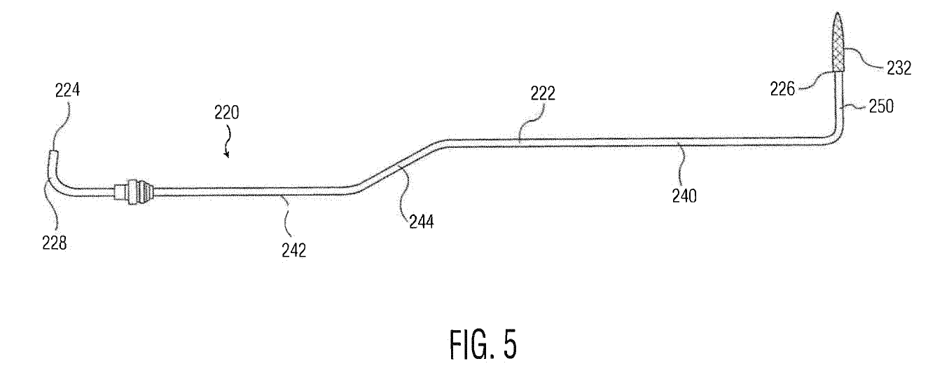

Referring to FIG. 5, still another embodiment of a perfusion apparatus 220 made in accordance with the present disclosure is shown. The perfusion apparatus 220 includes a hollow tubular member 222 including a first end 224 and a second and opposite end 226. A filter member 232 is attached to the second end 226 of the hollow tubular member 222. The hollow tubular member 222 includes a first straight section 250, a second straight second 242, and an angular section 244 positioned in between the first straight section 240 and the second straight section 242. The perfusion apparatus 220 further includes a first angular member 228 positioned at the first end 224 of the hollow tubular member 222.

In the embodiment illustrated in FIG. 5, the perfusion apparatus 220 further includes a second angular member 250 positioned at the second end 226 of the hollow tubular member 222. The second angular member 250 is for positioning the filter member 232 adjacent to the bottom of the bioreactor 10. For instance, the second angular member 250 can form an angle with the first straight section 240 of generally greater than about 40°, such as greater than about 50°, such as greater than about 60°, such as greater than about 70°, such as greater than about 80° and generally less than about 120°, such as less than about 100. For instance, as shown in FIG. 5, in one embodiment, the second angular member 250 forms a right angle with the first straight section 240 of the hollow tubular member 222. In this manner, the perfusion apparatus 220 can be placed in a bioreactor for avoiding interference with an impeller. The second angular member 250, on the other hand, allows for the filter member 232 to extend along the bottom of the bioreactor towards the center of the bioreactor or towards the wall of the bioreactor depending upon the particular application. Thus, the second angular member 250 can have a length suitable to place the filter member 232 at a desired location. The length of the second angular member 250, for instance, in one embodiment, can be generally greater than about 20 mm, such as greater than about 30 mm, such as greater than about 40 mm, such as greater than about 50 mm, such as greater than about 60 mm, such as greater than about 70 mm, such as greater than about 80 mm, such as greater than about 90 mm, such as greater than about 100 mm and generally less than about 500 mm, such as less than about 300 mm, such as less than about 200 mm, such as less than about 180 mm, such as less than about 160 mm, such as less than about 140 mm. The length of the second angular member 250, however, can depend upon the size and volume of the bioreactor 10. Thus, the length can be greater than or less than the dimensions provided above.

Referring to FIG. 6, yet another embodiment of a bioreactor system made in accordance with the present disclosure is shown. The bioreactor system includes a bioreactor 310 having a port 318 located on a side wall of the bioreactor. The bioreactor system further includes a perfusion apparatus 320 having a hollow tubular member 322 and a filter member 332. The perfusion apparatus 320 can be inserted into the port 318. The filter member 332 is similar to that as shown in greater detail in FIGS. 4A and 4B. The perfusion apparatus 320 may minimize the amount of space occupied in a bioreactor, which in some embodiments may allow the filter member 332 to include a longer mesh having a greater surface area. Allowing perfusion apparatus 320 access into the bioreactor 310 at the bottom side wall reduces the overall amount of material penetrating into the bioreactor 310, as shown in FIG. 6, compared to embodiments of the perfusion apparatus that are inserted through a port in the top of the bioreactor, for example as shown in FIG. 1.

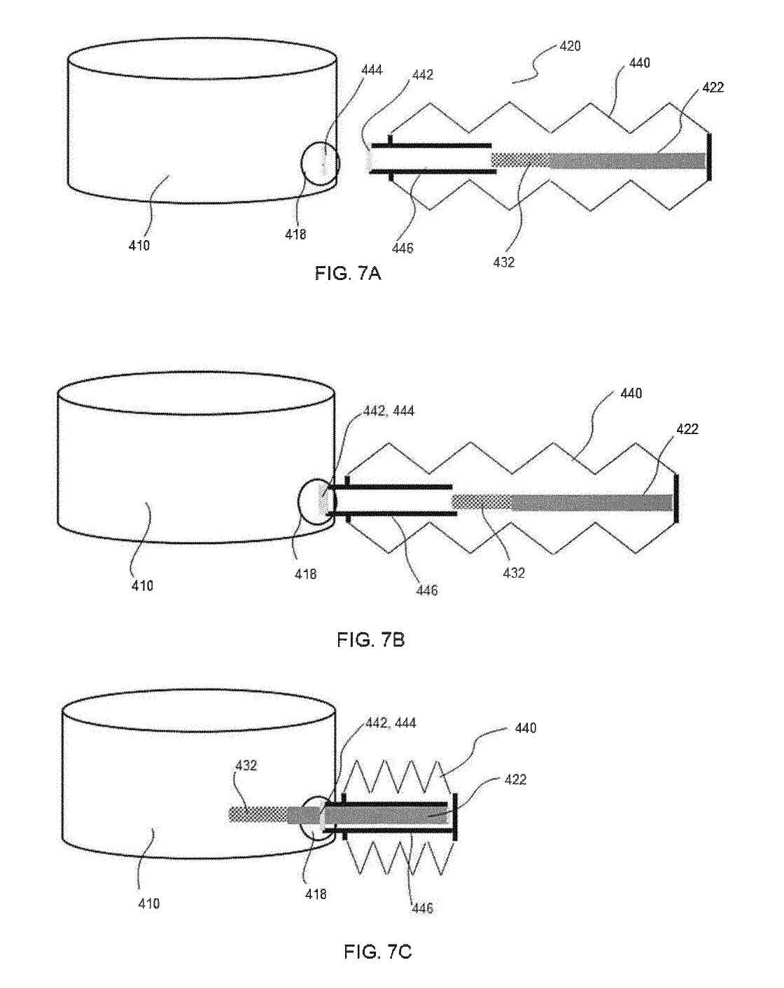

Referring now to FIGS. 7A to 7C, an additional embodiment of a bioreactor system made in accordance with the present disclosure is shown. The bioreactor system includes a bioreactor 410 having a port 418 located on a lower side wall of the bioreactor. The embodiment of FIGS. 7A to 7C further includes a perfusion apparatus 420 having a hollow tubular member 422 and a filter member 432.

In the embodiment shown in FIGS. 7A to 7C, the perfusion apparatus 420 further includes a collapsible bellows structure 440 for completely closed, sterile entry. The bellows 420 may be plastic. The hollow tubular structure 422 and the filter member 432 are completely encased in the bellows 440. The bellows 440 forms an enclosed environment that can be sterilized for containing the hollow tubular structure 422 and the filter member 432. The perfusion apparatus 420 further includes a rigid tunnel 446 within the bellows 440 leading to a sterile connection port 442. The sterile connection port 442 may be any commercially available sterile connection port that is compatible with the bioreactor 410. For example, the sterile connection port may be a Kleenpak™ Sterile Connector manufactured by Pall Biotech, an Opta® sterile connector manufactured by Sartorius, a ReadyMate single-use connector manufactured by GE Healthcare Life Sciences, or other commercially available sterile connector. The bioreactor 410 includes a matching sterile connector 444 in the port 418 on the bioreactor wall.

As shown in FIG. 7B, the sterile connections 442 and 444 of the perfusion apparatus 420 and bioreactor 410 are first connected to each other. A seal is formed between the sterile connections 442 and 444. Then, as shown in FIG. 7C, an opening is formed between the sterile connections 442 and 444. The bellows 440 can then be collapsed and the hollow tubular member 422 can be pushed through into the bioreactor 410, extending the filter member 432 into the bioreactor 410. The bellows 440 is collapsed when the hollow tubular member 422 and filter member 432 are pushed into the bioreactor.

Referring to FIGS. 8A and 8B, still another embodiment of a bioreactor system made in accordance with the present disclosure is shown. The bioreactor system includes a bioreactor 510 having a cone-shaped perfusion apparatus 520. The filter member 532 of the perfusion apparatus 520 is formed as a mesh patch on the wall 511 of the bioreactor 510. The mesh patch may be located on a side wall 511 of the bioreactor 510 as shown in FIG. 8B. The perfusion apparatus 520 has an enclosed volume 534 formed by a cone 536 that leads from the filter member 532 to an outlet hollow tubular member 522. In some embodiments, the cone 536 may be flexible.

Referring to FIG. 9, an additional embodiment of the bioreactor system made in accordance with the present disclosure is shown. The bioreactor system includes a bioreactor 610 having a cone-shaped perfusion apparatus 620 that can serve as a filtered drain for the bioreactor 610. The filter member 632 of the perfusion apparatus 620 is formed as a mesh patch on the bottom wall of the bioreactor 610. The perfusion apparatus 620 has an enclosed volume 634 formed by a cone 636 that leads from the filter member 632 to an outlet hollow tubular member 622. In some embodiments, the cone 636 may be flexible. The design of the embodiment shown in FIG. 9 allows the maximum amount of liquid to be drained out of the bioreactor, leaving just the microcarriers in the bioreactor. An agitator such as an impeller 16 as shown in FIG. 1 may be included in the bioreactor 610 in order to prevent microcarriers from settling on the mesh patch and clogging the filter member 632.

### Example

The following test was conducted in order to demonstrate the accuracy of perfusion performed using a perfusion apparatus in a bioreactor system made in accordance with an embodiment of the present disclosure. In each test, a bioreactor system was set up including a perfusion apparatus inserted into a bioreactor containing fluid media, microcarriers, and mesenchymal stem cells. Within a sterile hood, the perfusion apparatus was inserted into the bioreactor through a port. A controller for the perfusion apparatus was set to control a pump connected to the perfusion apparatus with a target rate of perfusion for a period of time, and perfusion was initiated. At the end of the test time period, a waste bag containing the fluid media perfused from the bioreactor was measured to determine if the target rate of perfusion was achieved. The performance of the perfusion apparatus was measured as a perfusion rate percentage relative to the target rate. On the y-axis, 100% perfusion rate relative to the target rate indicates that the perfusion was exactly on target. Perfusion of less fluid media than the target rate could indicate, for example, that the filter member was clogged by microcarriers. As can be seen by the data, the perfusion apparatus consistently delivered accuracy of within 10% on both a small scale (3 L bioreactor volume) and large scale (50 L bioreactor volume).

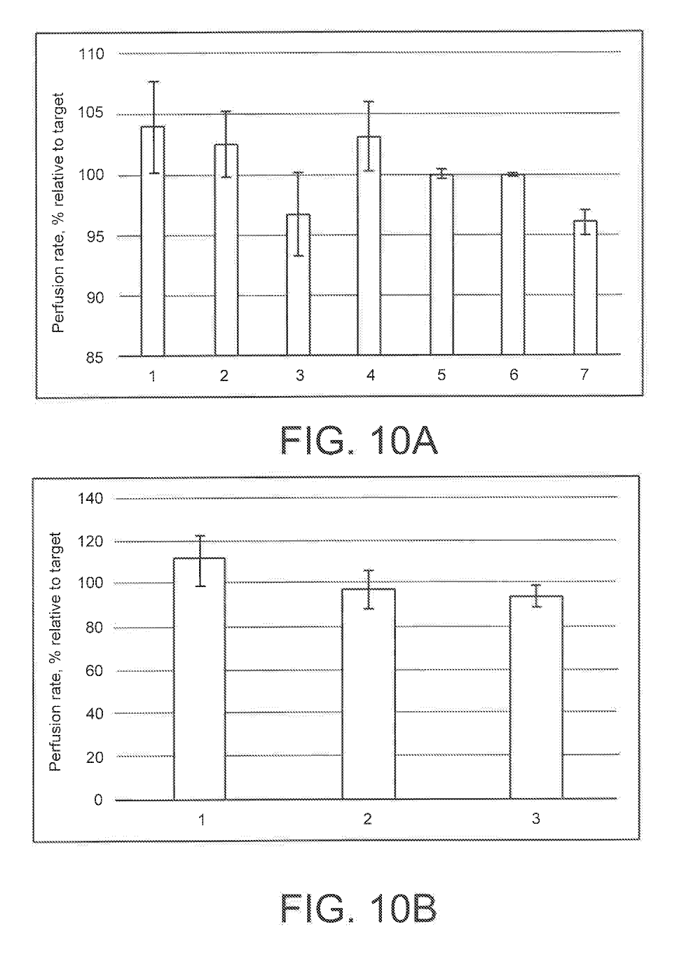

FIG. 10A shows the results of tests performed with a perfusion apparatus as illustrated by FIG. 2 and a bioreactor having a bioreactor volume of 3 L. This test was run seven times. Individual results from each test run are shown in FIG. 10A. As can be seen in FIG. 10A, the perfusion apparatus delivered within about 10% of the set point in each test run.

FIG. 10B shows the results of tests performed with a perfusion apparatus as illustrated by FIG. 3 and a bioreactor having a bioreactor volume of 50 L. This test was run three times. Individual results from each test run are shown in FIG. 10B. As can be seen in FIG. 10B, the perfusion apparatus delivered within 10% of the set point (50 L) in each test run.

The devices, facilities and methods described herein are suitable for use in and with culturing any desired cell line including prokaryotic and/or eukaryotic cell lines. Further, in embodiments, the devices, facilities and methods are suitable for culturing suspension cells or anchorage-dependent (adherent) cells and are suitable for production operations configured for production of pharmaceutical and biopharmaceutical products-such as polypeptide products, nucleic acid products (for example DNA or RNA), or cells and/or viruses such as those used in cellular and/or viral therapies.

In embodiments, the cells express or produce a product, such as a recombinant therapeutic or diagnostic product. As described in more detail below, examples of products produced by cells include, but are not limited to, antibody molecules (e.g., monoclonal antibodies, bispecific antibodies), antibody mimetics (polypeptide molecules that bind specifically to antigens but that are not structurally related to antibodies such as e.g. DARPins, affibodies, adnectins, or IgNARs), fusion proteins (e.g., Fc fusion proteins, chimeric cytokines), other recombinant proteins (e.g., glycosylated proteins, enzymes, hormones), viral therapeutics (e.g., anti-cancer oncolytic viruses, viral vectors for gene therapy and viral immunotherapy), cell therapeutics (e.g., pluripotent stem cells, mesenchymal stem cells and adult stem cells), vaccines or lipid-encapsulated particles (e.g., exosomes, virus-like particles), RNA (such as e.g. siRNA) or DNA (such as e.g. plasmid DNA), antibiotics or amino acids. In embodiments, the devices, facilities and methods can be used for producing biosimilars.

As mentioned, in embodiments, devices, facilities and methods allow for the production of eukaryotic cells, e.g., mammalian cells or lower eukaryotic cells such as for example yeast cells or filamentous fungi cells, or prokaryotic cells such as Gram-positive or Gram-negative cells and/or products of the eukaryotic or prokaryotic cells, e.g., proteins, peptides, antibiotics, amino acids, nucleic acids (such as DNA or RNA), synthesised by the eukaryotic cells in a large-scale manner. Unless stated otherwise herein, the devices, facilities, and methods can include any desired volume or production capacity including but not limited to bench-scale, pilot-scale, and full production scale capacities.

Moreover and unless stated otherwise herein, the devices, facilities, and methods can include any suitable reactor(s) including but not limited to stirred tank, airlift, fiber, microfiber, hollow fiber, ceramic matrix, fluidized bed, fixed bed, and/or spouted bed bioreactors. As used herein, “reactor” can include a fermentor or fermentation unit, or any other reaction vessel and the term “reactor” is used interchangeably with “fermentor.” For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO2 levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation processes. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass.

In embodiments and unless stated otherwise herein, the devices, facilities, and methods described herein can also include any suitable unit operation and/or equipment not otherwise mentioned, such as operations and/or equipment for separation, purification, and isolation of such products. Any suitable facility and environment can be used, such as traditional stick-built facilities, modular, mobile and temporary facilities, or any other suitable construction, facility, and/or layout. For example, in some embodiments modular clean-rooms can be used. Additionally and unless otherwise stated, the devices, systems, and methods described herein can be housed and/or performed in a single location or facility or alternatively be housed and/or performed at separate or multiple locations and/or facilities.

By way of non-limiting examples and without limitation, U.S. Publication Nos. 2013/0280797; 2012/0077429; 2011/0280797; 2009/0305626; and U.S. Pat. Nos. 8,298,054; 7,629,167; and 5,656,491, which are hereby incorporated by reference in their entirety, describe example facilities, equipment, and/or systems that may be suitable.

In embodiments, the cells are eukaryotic cells, e.g., mammalian cells. The mammalian cells can be for example human or rodent or bovine cell lines or cell strains. Examples of such cells, cell lines or cell strains are e.g. mouse myeloma (NSO)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EBI, EB2, EB3, oncolytic or hybridoma-cell lines. Preferably the mammalian cells are CHO-cell lines. In one embodiment, the cell is a CHO cell. In one embodiment, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the Potelligent® CHOK1 SV (Lonza Biologics, Inc.). Eukaryotic cells can also be avian cells, cell lines or cell strains, such as for example, EBx® cells, EB14, EB24, EB26, EB66, or EBvl3.

In one embodiment, the eukaryotic cells are stem cells. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs).

In one embodiment, the cell is a differentiated form of any of the cells described herein. In one embodiment, the cell is a cell derived from any primary cell in culture.

In embodiments, the cell is a hepatocyte such as a human hepatocyte, animal hepatocyte, or a non-parenchymal cell. For example, the cell can be a plateable metabolism qualified human hepatocyte, a plateable induction qualified human hepatocyte, plateable Qualyst Transporter Certified™ human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57Bl/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes). Example hepatocytes are commercially available from Triangle Research Labs, LLC, 6 Davis Drive Research Triangle Park, North Carolina, USA 27709.

In one embodiment, the eukaryotic cell is a lower eukaryotic cell such as e.g. a yeast cell (e.g., Pichia genus (e.g. Pichia pastoris, Pichia methanolica, Pichia kluyveri, and Pichia angusta), Komagataella genus (e.g. Komagataella pastoris, Komagataella pseudopastoris or Komagataella phaffii), Saccharomyces genus (e.g. Saccharomyces cerevisae, cerevisiae, Saccharomyces kluyveri, Saccharomyces uvarum), Kluyveromyces genus (e.g. Kluyveromyces lactis, Kluyveromyces marxianus), the Candida genus (e.g. Candida utilis, Candida cacaoi, Candida boidinii,), the Geotrichum genus (e.g. Geotrichum fermentans), Hansenula polymorpha, Yarrowia lipolytica, or Schizosaccharomyces pombe. Preferred is the species Pichia pastoris. Examples for Pichia pastoris strains are X33, GS115, KM71, KM71H; and CBS7435.

In one embodiment, the eukaryotic cell is a fungal cell (e.g. Aspergillus (such as A. niger, A. fumigatus, A. orzyae, A. nidula), Acremonium (such as A. thermophilum), Chaetomium (such as C. thermophilum), Chrysosporium (such as C. thermophile), Cordyceps (such as C. militaris), Corynascus, Ctenomyces, Fusarium (such as F. oxysporum), Glomerella (such as G. graminicola), Hypocrea (such as H. jecorina), Magnaporthe (such as M. orzyae), Myceliophthora (such as M. thermophile), Nectria (such as N. heamatococca), Neurospora (such as N. crassa), Penicillium, Sporotrichum (such as S. thermophile), Thielavia (such as T. terrestris, T. heterothallica), Trichoderma (such as T. reesei), or Verticillium (such as V. dahlia)).

In one embodiment, the eukaryotic cell is an insect cell (e.g., Sf9, Mimic™ Sf9, Sf21, High Five™ (BT1-TN-5B1-4), or BT1-Ea88 cells), an algae cell (e.g., of the genus Amphora, Bacillariophyceae, Dunaliella, Chlorella, Chlamydomonas, Cyanophyta (cyanobacteria), Nannochloropsis, Spirulina, or Ochromonas), or a plant cell (e.g., cells from monocotyledonous plants (e.g., maize, rice, wheat, or Setaria), or from a dicotyledonous plants (e.g., cassava, potato, soybean, tomato, tobacco, alfalfa, Physcomitrella patens or Arabidopsis).

In one embodiment, the cell is a bacterial or prokaryotic cell.

In embodiments, the prokaryotic cell is a Gram-positive cells such as Bacillus, Streptomyces Streptococcus, Staphylococcus or Lactobacillus. Bacillus that can be used is, e.g. the B. subtilis, B. amyloliquefaciens, B. licheniformis, B. natto, or B. megaterium. In embodiments, the cell is B. subtilis, such as B. subtilis 3NA and B. subtilis 168. Bacillus is obtainable from, e.g., the Bacillus Genetic Stock Center, Biological Sciences 556, 484 West 12^{th} Avenue, Columbus Ohio 43210-1214.

In one embodiment, the prokaryotic cell is a Gram-negative cell, such as Salmonella spp. or Escherichia coli, such as e.g., TG1, TG2, W3110, DH1, DHB4, DH5a, HMS 174, HMS174 (DE3), NM533, C600, HB101, JM109, MC4100, XL1-Blue and Origami, as well as those derived from E. coli B-strains, such as for example BL-21 or BL21 (DE3), all of which are commercially available.

Suitable host cells are commercially available, for example, from culture collections such as the DSMZ (Deutsche Sammlung von Mikroorganismen and Zellkulturen GmbH, Braunschweig, Germany) or the American Type Culture Collection (ATCC).

In embodiments, the cultured cells are used to produce proteins e.g., antibodies, e.g., monoclonal antibodies, and/or recombinant proteins, for therapeutic use. In embodiments, the cultured cells produce peptides, amino acids, fatty acids or other useful biochemical intermediates or metabolites. For example, in embodiments, molecules having a molecular weight of about 4000 daltons to greater than about 140,000 daltons can be produced. In embodiments, these molecules can have a range of complexity and can include posttranslational modifications including glycosylation.

In embodiments, the protein is, e.g., BOTOX, Myobloc, Neurobloc, Dysport (or other serotypes of botulinum neurotoxins), alglucosidase alpha, daptomycin, YH-16, choriogonadotropin alpha, filgrastim, cetrorelix, interleukin-2, aldesleukin, teceleulin, denileukin diftitox, interferon alpha-n3 (injection), interferon alpha-ni, DL-8234, interferon, Suntory (gamma-1a), interferon gamma, thymosin alpha 1, tasonermin, DigiFab, ViperaTAb, EchiTAb, CroFab, nesiritide, abatacept, alefacept, Rebif, eptoterminalfa, teriparatide (osteoporosis), calcitonin injectable (bone disease), calcitonin (nasal, osteoporosis), etanercept, hemoglobin glutamer 250 (bovine), drotrecogin alpha, collagenase, carperitide, recombinant human epidermal growth factor (topical gel, wound healing), DWP401, darbepoetin alpha, epoetin omega, epoetin beta, epoetin alpha, desirudin, lepirudin, bivalirudin, nonacog alpha, Mononine, eptacog alpha (activated), recombinant Factor VIII+VWF, Recombinate, recombinant Factor VIII, Factor VIII (recombinant), Alphnmate, octocog alpha, Factor VIII, palifermin, Indikinase, tenecteplase, alteplase, pamiteplase, reteplase, nateplase, monteplase, follitropin alpha, rFSH, hpFSH, micafungin, pegfilgrastim, lenograstim, nartograstim, sermorelin, glucagon, exenatide, pramlintide, iniglucerase, galsulfase, Leucotropin, molgramostim, triptorelin acetate, histrelin (subcutaneous implant, Hydron), deslorelin, histrelin, nafarelin, leuprolide sustained release depot (ATRIGEL), leuprolide implant (DUROS), goserelin, Eutropin, KP-102 program, somatropin, mecasermin (growth failure), enlfavirtide, Org-33408, insulin glargine, insulin glulisine, insulin (inhaled), insulin lispro, insulin detemir, insulin (buccal, RapidMist), mecasermin rinfabate, anakinra, celmoleukin, 99 mTc-apcitide injection, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex' recombinant human luteinizing hormone, domase alpha, trafermin, ziconotide, taltirelin, diboterminalfa, atosiban, becaplermin, eptifibatide, Zemaira, CTC-111, Shanvac-B, HPV vaccine (quadrivalent), octreotide, lanreotide, ancestim, agalsidase beta, agalsidase alpha, laronidase, prezatide copper acetate (topical gel), rasburicase, ranibizumab, Actimmune, PEG-Intron, Tricomin, recombinant house dust mite allergy desensitization injection, recombinant human parathyroid hormone (PTH) 1-84 (sc, osteoporosis), epoetin delta, transgenic antithrombin III, Granditropin, Vitrase, recombinant insulin, interferon-alpha (oral lozenge), GEM-21S, vapreotide, idursulfase, omnapatrilat, recombinant serum albumin, certolizumab pegol, glucarpidase, human recombinant C1 esterase inhibitor (angioedema), lanoteplase, recombinant human growth hormone, enfuvirtide (needle-free injection, Biojector 2000), VGV-1, interferon (alpha), lucinactant, aviptadil (inhaled, pulmonary disease), icatibant, ecallantide, omiganan, Aurograb, pexigananacetate, ADI-PEG-20, LDI-200, degarelix, cintredelinbesudotox, Favld, MDX-1379, ISAtx-247, liraglutide, teriparatide (osteoporosis), tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone (sustained release injection), recombinant G-CSF, insulin (inhaled, AIR), insulin (inhaled, Technosphere), insulin (inhaled, AERx), RGN-303, DiaPep277, interferon beta (hepatitis C viral infection (HCV)), interferon alpha-n3 (oral), belatacept, transdermal insulin patches, AMG-531, MBP-8298, Xerecept, opebacan, AIDSVAX, GV-1001, LymphoScan, ranpirnase, Lipoxysan, lusupultide, MP52 (beta-tricalciumphosphate carrier, bone regeneration), melanoma vaccine, sipuleucel-T, CTP-37, Insegia, vitespen, human thrombin (frozen, surgical bleeding), thrombin, TransMID, alfimeprase, Puricase, terlipressin (intravenous, hepatorenal syndrome), EUR-1008M, recombinant FGF-I (injectable, vascular disease), BDM-E, rotigaptide, ETC-216, P-113, MBI-594AN, duramycin (inhaled, cystic fibrosis), SCV-07, OPI-45, Endostatin, Angiostatin, ABT-510, Bowman Birk Inhibitor Concentrate, XMP-629, 99 mTc-Hynic-Annexin V, kahalalide F, CTCE-9908, teverelix (extended release), ozarelix, romidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, ovarian cancer immunotherapeutic vaccine, SB-249553, Oncovax-CL, OncoVax-P, BLP-25, CerVax-16, multi-epitope peptide melanoma vaccine (MART-1, gp100, tyrosinase), nemifitide, rAAT (inhaled), rAAT (dermatological), CGRP (inhaled, asthma), pegsunercept, thymosinbeta4, plitidepsin, GTP-200, ramoplanin, GRASPA, OBI-1, AC-100, salmon calcitonin (oral, eligen), calcitonin (oral, osteoporosis), examorelin, capromorelin, Cardeva, velafermin, 131I-TM-601, KK-220, T-10, ularitide, depelestat, hematide, Chrysalin (topical), rNAPc2, recombinant Factor V111 (PEGylated liposomal), bFGF, PEGylated recombinant staphylokinase variant, V-10153, SonoLysis Prolyse, NeuroVax, CZEN-002, islet cell neogenesis therapy, rGLP-1, BIM-51077, LY-548806, exenatide (controlled release, Medisorb), AVE-0010, GA-GCB, avorelin, ACM-9604, linaclotid eacetate, CETi-1, Hemospan, VAL (injectable), fast-acting insulin (injectable, Viadel), intranasal insulin, insulin (inhaled), insulin (oral, eligen), recombinant methionyl human leptin, pitrakinra subcutancous injection, eczema), pitrakinra (inhaled dry powder, asthma), Multikine, RG-1068, MM-093, NBI-6024, AT-001, PI-0824, Org-39141, Cpn10 (autoimmune diseases/inflammation), talactoferrin (topical), rEV-131 (ophthalmic), rEV-131 (respiratory disease), oral recombinant human insulin (diabetes), RPI-78M, oprelvekin (oral), CYT-99007 CTLA4-Ig, DTY-001, valategrast, interferon alpha-n3 (topical), IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, malaria vaccine (virosomes, PeviPRO), ALTU-135, parvovirus B19 vaccine, influenza vaccine (recombinant neuraminidase), malaria/HBV vaccine, anthrax vaccine, Vacc-5q, Vacc-4x, HIV vaccine (oral), HPV vaccine, Tat Toxoid, YSPSL, CHS-13340, PTH(1-34) liposomal cream (Novasome), Ostabolin-C, PTH analog (topical, psoriasis), MBRI-93.02, MTB72F vaccine (tuberculosis), MVA-Ag85A vaccine (tuberculosis), FARA04, BA-210, recombinant plague FIV vaccine, AG-702, OxSODrol, rBetV1, Der-p1/Der-p2/Der-p7 allergen-targeting vaccine (dust mite allergy), PR1 peptide antigen (leukemia), mutant ras vaccine, HPV-16 E7 lipopeptide vaccine, labyrinthin vaccine (adenocarcinoma), CML vaccine, WT1-peptide vaccine (cancer), IDD-5, CDX-110, Pentrys, Norelin, CytoFab, P-9808, VT-111, icrocaptide, telbermin (dermatological, diabetic foot ulcer), rupintrivir, reticulose, rGRF, HA, alpha-galactosidase A, ACE-011, ALTU-140, CGX-1160, angiotensin therapeutic vaccine, D-4F, ETC-642, APP-018, rhMBL, SCV-07 (oral, tuberculosis), DRF-7295, ABT-828, ErbB2-specific immunotoxin (anticancer), DT3SSIL-3, TST-10088, PRO-1762, Combotox, cholecystokinin-B/gastrin-receptor binding peptides, 111In-hEGF, AE-37, trasnizumab-DM1, Antagonist G, IL-12 (recombinant), PM-02734, IMP-321, rhIGF-BP3, BLX-883, CUV-1647 (topical), L-19 based radioimmunotherapeutics (cancer), Re-188-P-2045, AMG-386, DC/1540/KLH vaccine (cancer), VX-001, AVE-9633, AC-9301, NY-ESO-1 vaccine (peptides), NA17.A2 peptides, melanoma vaccine (pulsed antigen therapeutic), prostate cancer vaccine, CBP-501, recombinant human lactoferrin (dry eye), FX-06, AP-214, WAP-8294A (injectable), ACP-HIP, SUN-11031, peptide YY [3-36](obesity, intranasal), FGLL, atacicept, BR3-Fc, BN-003, BA-058, human parathyroid hormone 1-34 (nasal, osteoporosis), F-18-CCR1, AT-1100 (celiac disease/diabetes), JPD-003, PTH(7-34) liposomal cream (Novasome), duramycin (ophthalmic, dry eye), CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix (immediate release), EP-51216, hGH (controlled release, Biosphere), OGP-I, sifuvirtide, TV4710, ALG-889, Org-41259, rhCC10, F-991, thymopentin (pulmonary diseases), r(m)CRP, hepatoselective insulin, subalin, L19-IL-2 fusion protein, elafin, NMK-150, ALTU-139, EN-122004, rhTPO, thrombopoietin receptor agonist (thrombocytopenic disorders), AL-108, AL-208, nerve growth factor antagonists (pain), SLV-317, CGX-1007, INNO-105, oral teriparatide (eligen), GEM-OS1, AC-162352, PRX-302, LFn-p24 fusion vaccine (Therapore), EP-1043, S pneumoniae pediatric vaccine, malaria vaccine, Neisseria meningitidis Group B vaccine, neonatal group B streptococcal vaccine, anthrax vaccine, HCV vaccine (gpE1+gpE2+MF−59), otitis media therapy, HCV vaccine (core antigen+ISCOMATRIX), hPTH(1-34) (transdermal, ViaDerm), 768974, SYN-101, PGN-0052, aviscumnine, BIM-23190, tuberculosis vaccine, multi-epitope tyrosinase peptide, cancer vaccine, enkastim, APC-8024, GI-5005, ACC-001, TTS-CD3, vascular-targeted TNF (solid tumors), desmopressin (buccal controlled-release), onercept, and TP-9201.

In some embodiments, the polypeptide is adalimumab (HUMIRA), infliximab (REMICADE™), rituximab (RITUXAN™/MAB THERA™) etanercept (ENBREL™), bevacizumab (AVASTIN™), trastuzumab (HERCEPTIN™), pegrilgrastim (NEULASTA™), or any other suitable polypeptide including biosimilars and biobetters.

Other suitable polypeptides are those listed below and in Table 1 of US2016/0097074:

TABLE I

| Protein Product | Reference Listed Drug |
|---|---|
| interferon gamma-1b | Actimmune ® |
| alteplase; tissue plasminogen activator | Activase ®/Cathflo ® |
| Recombinant antihemophilic factor | Advate |
| human albumin | Albutein ® |
| Laronidase | Aldurazyme ® |
| Interferon alfa-N3, human leukocyte derived | Alferon N ® |
| human antihemophilic factor | Alphanate ® |
| virus-filtered human coagulation factor IX | AlphaNine ® SD |
| Alefacept; recombinant, dimeric fusion | Amevive ® |
| protein LFA3-Ig | |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| Interferon beta-1b | Betaseron ® |
| Tositumomab | BEXXAR ® |
| antihemophilic factor | Bioclate ™ |
| human growth hormone | BioTropin ™ |
| botulinum toxin type A | BOTOX ® |
| Alemtuzumab | Campath ® |
| acritumomab; technetium-99 labeled | CEA-Scan ® |
| alglucerase; modified form of beta- | Ceredase ® |
| glucocerebrosidase | |
| imiglucerase; recombinant form of beta- | Cerezyme ® |
| glucocerebrosidase | |
| crotalidae polyvalent immune Fab, ovine | CroFab ™ |
| digoxin immune fab [ovine] | DigiFab ™ |
| Rasburicase | Elitek ® |
| Etanercept | ENBREL ® |
| epoietin alfa | Epogen ® |
| Cetuximab | Erbitux ™ |
| algasidase beta | Fabrazyme ® |
| Urofollitropin | Fertinex ™ |
| follitropin beta | Follistim ™ |
| Teriparatide | FORTEO ® |
| human somatropin | GenoTropin ® |
| Glucagon | GlucaGen ® |
| follitropin alfa | Gonal-F ® |
| antihemophilic factor | Helixate ® |
| Antihemophilic Factor; Factor XIII | HEMOFIL |
| adefovir dipivoxil | Hepsera ™ |
| Trastuzumab | Herceptin ® |
| Insulin | Humalog ® |
| antihemophilic factor/von Willebrand factor | Humate-P ® |
| complex-human | |
| Somatotropin | Humatrope ® |
| Adalimumab | HUMIRA ™ |
| human insulin | Humulin ® |
| recombinant human hyaluronidase | Hylenex ™ |
| interferon alfacon-1 | Infergen ® |
| eptifibatide | Integrilin ™ |
| alpha-interferon | Intron A ® |
| Palifermin | Kepivance |
| Anakinra | Kineret ™ |
| antihemophilic factor | Kogenate ® FS |
| insulin glargine | Lantus ® |
| granulocyte macrophage colony-stimulating factor | Leukine ®/Leukine ® Liquid |
| lutropin alfa for injection | Luveris |
| OspA lipoprotein | LYMErix ™ |
| Ranibizumab | LUCENTIS ® |
| gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesomab | NeutroSpec ™ (formerly |
| | LeuTech ®) |
| somatropin [rDNA] | Norditropin ®/Norditropin |
| | Nordiflex ® |
| Mitoxantrone | Novantrone ® |
| insulin; zinc suspension; | Novolin L ® |
| insulin; isophane suspension | Novolin N ® |
| insulin, regular; | Novolin R ® |
| Insulin | Novolin ® |
| coagulation factor VIIa | NovoSeven ® |
| Somatropin | Nutropin ® |
| immunoglobulin intravenous | Octagam ® |
| PEG-L-asparaginase | Oncaspar ® |
| abatacept, fully human soluable fusion | Orencia ™ |
| protein | |
| muromomab-CD3 | Orthoclone OKT3 ® |
| high-molecular weight hyaluronan | Orthovisc ® |
| human chorionic gonadotropin | Ovidrel ® |
| live attenuated Bacillus Calmette-Guerin | Pacis ® |
| peginterferon alfa-2a | Pegasys ® |
| pegylated version of interferon alfa-2b | PEG-Intron ™ |
| Abarelix (injectable suspension); | Plenaxis ™ |
| gonadotropin-releasing hormone | |
| antagonist | |
| epoietin alfa | Procrit ® |
| Aldesleukin | Proleukin, IL-2 ® |
| Somatrem | Protropin ® |
| dornase alfa | Pulmozyme ® |
| Efalizumab; selective, reversible T-cell | RAPTIVA ™ |
| blocker | |
| combination of ribavirin and alpha interferon | Rebetron ™ |
| Interferon beta 1a | Rebif ® |
| antihemophilic factor | Recombinate ® rAHF/ |
| antihemophilic factor | ReFacto ® |
| Lepirudin | Refludan ® |
| Infliximab | REMICADE ® |
| Abciximab | ReoPro ™ |
| Reteplase | Retavase ™ |
| Rituxima | Rituxan ™ |
| interferon alfa-2^{a} | Roferon-A ® |
| Somatropin | Saizen ® |
| synthetic porcine secretin | SecreFlo ™ |
| Basiliximab | Simulect ® |
| Eculizumab | SOLIRIS (R) |
| Pegvisomant | SOMAVERT ® |
| Palivizumab; recombinantly produced, | Synagis ™ |
| humanized mAb | |
| thyrotropin alfa | Thyrogen ® |
| Tenecteplase | TNKase ™ |
| Natalizumab | TYSABRI ® |
| human immune globulin intravenous 5% | Venoglobulin-S ® |
| and 10% solutions | |
| interferon alfa-n1, lymphoblastoid | Wellferon ® |
| drotrecogin alfa | Xigris ™ |
| Omalizumab; recombinant DNA-derived | Xolair ® |
| humanized monoclonal | |
| antibody targeting immunoglobulin-E | |
| Daclizumab | Zenapax ® |
| ibritumomab tiuxetan | Zevalin ™ |
| Somatotropin | Zorbtive ™ (Serostim ®) |

In embodiments, the polypeptide is a hormone, blood clotting/coagulation factor, cytokine/growth factor, antibody molecule, fusion protein, protein vaccine, or peptide as shown in Table 2.

TABLE 2
Exemplary Products

| Therapeutic | | |
|---|---|---|
| Product type | Product | Trade Name |
| Hormone | Erythropoietin, Epoein-α | Epogen, Procrit |
| | Darbepoetin-α | Aranesp |
| | Growth hormone (GH), | Genotropin, Humatrope, Norditropin, |
| | somatotropin | NovIVitropin, Nutropin, Omnitrope, |
| | | Protropin, Siazen, Serostim, Valtropin |
| | Human follicle- | Gonal-F, Follistim |
| | stimulating hormone | |
| | (FSH) | Ovidrel |
| | Human chorionic | Luveris |
| | gonadotropin | GlcaGen |
| | Lutropin-α | Geref |
| | Glucagon | ChiRhoStim (human peptide), |
| | Growth hormone releasing | SecreFlo (porcine peptide) |
| | hormone (GHRH) | Thyrogen |
| | Secretin | |
| | Thyroid stimulating | |
| | hormone (TSH), | |
| | thyrotropin | |
| Blood | Factor VIIa | NovoSeven |
| Clotting/Coagulation | Factor VIII | Bioclate, Helixate, Kogenate, |
| Factors | | Recombinate, ReFacto |
| | Factor IX | |
| | Antithrombin III (AT-III) | Benefix |
| | Protein C concentrate | Thrombate III |
| | | Ceprotin |
| Cytokine/Growth | Type I alpha-interferon | Infergen |
| factor | Interferon-αn3 (IFNαn3) | Alferon N |
| | Interferon-β1a (rIFN- β) | Avonex, Rebif |
| | Interferon-β1b (rIFN- β) | Betaseron |
| | Interferon-γ1b (IFN γ) | Actimmune |
| | Aldesleukin (interleukin | Proleukin |
| | 2(IL2), epidermal | |
| | theymocyte activating | |
| | factor; ETAF | Kepivance |
| | Palifermin (keratinocyte | Regranex |
| | growth factor; KGF) | |
| | Becaplemin (platelet- | Anril, Kineret |
| | derived growth factor; | |
| | PDGF) | |
| | Anakinra (recombinant | |
| | IL1 antagonist) | |
| Antibody molecules | Bevacizumab (VEGFA | Avastin |
| | mAb) | Erbitux |
| | Cetuximab (EGFR mAb) | Vectibix |
| | Panitumumab (EGFR | Campath |
| | mAb) | Rituxan |
| | Alemtuzumab (CD52 | Herceptin |
| | mAb) | Orencia |
| | Rituximab (CD20 | Humira |
| | chimeric Ab) | Enbrel |
| | Trastuzumab (HER2/Neu | |
| | mAb) | Remicade |
| | Abatacept (CTLA Ab/Fc | Amevive |
| | fusion) | Raptiva |
| | Adalimumab | Tysabri |
| | (TNFα mAb) | Soliris |
| | Etanercept (TNF | Orthoclone, OKT3 |
| | receptor/Fc fusion) | |
| | Infliximab (TNFα | |
| | chimeric mAb) | |
| | Alefacept (CD2 fusion | |
| | protein) | |
| | Efalizumab (CD11a mAb) | |
| | Natalizumab (integrin α4 | |
| | subunit mAb) | |
| | Eculizumab (C5mAb) | |
| | Muromonab-CD3 | |
| Other: | Insulin | Humulin, Novolin |
| Fusion | Hepatitis B surface | Engerix, Recombivax HB |
| proteins/Protein | antigen (HBsAg) | |
| vaccines/Peptides | HPV vaccine | Gardasil |
| | OspA | LYMErix |
| | Anti-Rhesus(Rh) | Rhophylac |
| | immunoglobulin G | Fuzeon |
| | Enfuvirtide | |
| | Spider silk, e.g., fibrion | QMONOS |

In embodiments, the protein is multispecific protein, e.g., a bispecific antibody as shown in Table 3.

TABLE 3
Bispecific Formats

| Name (other | | | | | |
|---|---|---|---|---|---|
| names, | | | Proposed | | Diseases (or |
| sponsoring | BsAb | | mechanisms of | Development | healthy |
| organizations) | format | Targets | action | stages | volunteers) |
| Catumaxomab | BsIgG: | CD3, | Retargeting of T | Approved in | Malignant |
| (Removab ®, | Triomab | EpCAM | cells to tumor, Fc | EU | ascites in |
| Fresenius | | | mediated effector | | EpCAM |
| Biotech, Trion | | | functions | | positive tumors |
| Pharma, | | | | | |
| Neopharm) | | | | | |
| Ertumaxomab | BsIgG: | CD3, HER2 | Retargeting of T | Phase I/II | Advanced solid |
| (Neovii Biotech, | Triomab | | cells to tumor | | tumors |
| Fresenius | | | | | |
| Biotech) | | | | | |
| Blinatumomab | BiTE | CD3, CD19 | Retargeting of T | Approved in | Precursor B-cell |
| (Blincyto ®, AMG | | | cells to tumor | USA | ALL |
| 103, MT 103, | | | | Phase II and | ALL |
| MEDI 538, | | | | III | DLBCL |
| Amgen) | | | | Phase II | NHL |
| | | | | Phase I | |
| REGN1979 | BsAb | CD3, CD20 | | | |
| (Regeneron) | | | | | |
| Solitomab (AMG | BiTE | CD3, | Retargeting of T | Phase I | Solid tumors |
| 110, MT110, | | EpCAM | cells to tumor | | |
| Amgen) | | | | | |
| MEDI 565 | BiTE | CD3, CEA | Retargeting of T | Phase I | Gastrointestinal |
| (AMG 211, | | | cells to tumor | | adenocancinoma |
| MedImmune, | | | | | |
| Amgen) | | | | | |
| RO6958688 | BsAb | CD3, CEA | | | |
| (Roche) | | | | | |
| BAY2010112 | BiTE | CD3, PSMA | Retargeting of T | Phase I | Prostate cancer |
| (AMG 212, | | | cells to tumor | | |
| Bayer; Amgen) | | | | | |
| MGD006 | DART | CD3, CD123 | Retargeting of T | Phase I | AML |
| (Macrogenics) | | | cells to tumor | | |
| MGD007 | DART | CD3, gpA33 | Retargeting of T | Phase I | Colorectal |
| (Macrogenics) | | | cells to tumor | | cancer |
| MGD011 | DART | CD19, CD3 | | | |
| (Macrogenics) | | | | | |
| SCORPION | BsAb | CD3, CD19 | Retargeting of T | | |
| (Emergent | | | cells to tumor | | |
| Biosolutions, | | | | | |
| Trubion) | | | | | |
| AFM11 (Affimed | TandAb | CD3, CD19 | Retargeting of T | Phase I | NHL and ALL |
| Therapeutics) | | | cells to tumor | | |
| AFM12 (Affimed | TandAb | CD19, CD16 | Retargeting of | | |
| Therapeutics) | | | NK cells to | | |
| | | | tumor cells | | |
| AFM13 (Affimed | TandAb | CD30, | Retargeting of | Phase II | Hodgkin's |
| Therapeutics) | | CD16A | NK cells to | | Lymphoma |
| | | | tumor cells | | |
| GD2 (Barbara | T cells | CD3, GD2 | Retargeting of T | Phase I/II | Neuroblastoma |
| Ann Karmanos | preloaded | | cells to tumor | | and |
| Cancer Institute) | with BsAb | | | | osteosarcoma |
| pGD2 (Barbara | T cells | CD3, Her2 | Retargeting of T | Phase II | Metastatic |
| Ann Karmanos | preloaded | | cells to tumor | | breast cancer |
| Cancer Institute) | with BsAb | | | | |
| EGFRBi-armed | T cells | CD3, EGFR | Autologous | Phase I | Lung and other |
| autologous | preloaded | | activated T cells | | solid tumors |
| activated T cells | with BsAb | | to EGFR-positive | | |
| (Roger Williams | | | tumor | | |
| Medical Center) | | | | | |
| Anti-EGFR- | T cells | CD3, EGFR | Autologous | Phase I | Colon and |
| armed activated | preloaded | | activated T cells | | pancreatic |
| T-cells (Barbara | with BsAb | | to EGFR-positive | | cancers |
| Ann Karmanos | | | tumor | | |
| Cancer Institute) | | | | | |
| rM28 (University | Tandem | CD28, | Retargeting of T | Phase II | Metastatic |
| Hospital | scFv | MAPG | cells to tumor | | melanoma |
| Tübingen) | | | | | |
| IMCgp100 | ImmTAC | CD3, peptide | Retargeting of T | Phase I/II | Metastatic |
| (Immunocore) | | MHC | cells to tumor | | melanoma |
| DT2219ARL | 2 scFv | CD19, CD22 | Targeting of | Phase I | B cell leukemia |
| (NCI, University | linked to | | protein toxin to | | or lymphoma |
| of Minnesota) | diphtheria | | tumor | | |
| | toxin | | | | |
| XmAb5871 | BsAb | CD19, | | | |
| (Xencor) | | CD32b | | | |
| NI-1701 | BsAb | CD47, CD19 | | | |
| (NovImmune) | | | | | |
| MM-111 | BsAb | ErbB2, | | | |
| (Merrimack) | | ErbB3 | | | |
| MM-141 | BsAb | IGF-1R, | | | |
| (Merrimack) | | ErbB3 | | | |
| NA (Merus) | BsAb | HER2, | | | |
| | | HER3 | | | |
| NA (Merus) | BsAb | CD3, | | | |
| | | CLEC12A | | | |
| NA (Merus) | BsAb | EGFR, | | | |
| | | HER3 | | | |
| NA (Merus) | BsAb | PD1, | | | |
| | | undisclosed | | | |
| NA (Merus) | BsAb | CD3, | | | |
| | | undisclosed | | | |
| Duligotuzumab | DAF | EGFR, | Blockade of 2 | Phase I and II | Head and neck |
| (MEHD7945A, | | HER3 | receptors, ADCC | Phase II | cancer |
| Genentech, | | | | | Colorectal |
| Roche) | | | | | cancer |
| LY3164530 (Eli | Not | EGFR, MET | Blockade of 2 | Phase I | Advanced or |
| Lily) | disclosed | | receptors | | metastatic |
| | | | | | cancer |
| MM-111 | HSA body | HER2, | Blockade of 2 | Phase II | Gastric and |
| (Merrimack | | HER3 | receptors | Phase I | esophageal |
| Pharmaceuticals) | | | | | cancers |
| | | | | | Breast cancer |
| MM-141, | IgG-scFv | IGF-1R, | Blockade of 2 | Phase I | Advanced solid |
| (Merrimack | | HER3 | receptors | | tumors |
| Pharmaceuticals) | | | | | |
| RG7221 | CrossMab | Ang2, | Blockade of 2 | Phase I | Solid tumors |
| (RO5520985, | | VEGF A | proangiogenics | | |
| Roche) | | | | | |
| RG7716 (Roche) | CrossMab | Ang2, | Blockade of 2 | Phase I | Wet AMD |
| | | VEGF A | proangiogenics | | |
| OMP-305B83 | BsAb | DLL4/VEGF | | | |
| (OncoMed) | | | | | |
| TF2 | Dock and | CEA, HSG | Pretargeting | Phase II | Colorectal, |
| (Immunomedics) | lock | | tumor for PET or | | breast and lung |
| | | | radioimaging | | cancers |
| ABT-981 | DVD-Ig | IL-1α, IL-1β | Blockade of 2 | Phase II | Osteoarthritis |
| (AbbVie) | | | proinflammatory | | |
| | | | cytokines | | |
| ABT-122 | DVD-Ig | TNF, IL- | Blockade of 2 | Phase II | Rheumatoid |
| (AbbVie) | | 17A | proinflammatory | | arthritis |
| | | | cytokines | | |
| COVA322 | IgG- | TNF, IL17A | Blockade of 2 | Phase I/II | Plaque psoriasis |
| | fynomer | | proinflammatory | | |
| | | | cytokines | | |
| SAR156597 | Tetravalent | IL-13, IL-4 | Blockade of 2 | Phase I | Idiopathic |
| (Sanofi) | bispecific | | proinflammatory | | pulmonary |
| | tandem | | cytokines | | fibrosis |
| | IgG | | | | |
| GSK2434735 | Dual- | IL-13, IL-4 | Blockade of 2 | Phase I | (Healthy |
| (GSK) | targeting | | proinflammatory | | volunteers) |
| | domain | | cytokines | | |
| Ozoralizumab | Nanobody | TNF, HSA | Blockade of | Phase II | Rheumatoid |
| (ATN103, | | | proinflammatory | | arthritis |
| Ablynx) | | | cytokine, binds to | | |
| | | | HSA to increase | | |
| | | | half-life | | |
| ALX-0761 | Nanobody | IL-17A/F, | Blockade of 2 | Phase I | (Healthy |
| (Merck Serono, | | HSA | proinflammatory | | volunteers) |
| Ablynx) | | | cytokines, binds | | |
| | | | to HSA to | | |
| | | | increase half-life | | |
| ALX-0061 | Nanobody | IL-6R, HSA | Blockade of | Phase I/II | Rheumatoid |
| (AbbVie, | | | proinflammatory | | arthritis |
| Ablynx; | | | cytokine, binds to | | |
| | | | HSA to increase | | |
| | | | half-life | | |
| ALX-0141 | Nanobody | RANKL, | Blockade of bone | Phase I | Postmenopausal |
| (Ablynx, | | HSA | resorption, binds | | bone loss |
| Eddingpharm) | | | to HSA to | | |
| | | | increase half-life | | |
| RG6013/ACE910 | ART-Ig | Factor IXa, | Plasma | Phase II | Hemophilia |
| (Chugai, Roche) | | factor X | coagulation | | |

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A perfusion apparatus comprising:
a hollow tubular member for perfusing fluid from a bioreactor, the hollow tubular member having a first end defining a first opening and a second end opposite end defining a second opening, the second opening having a cross-sectional area: and
a filter member located and attached to the second end of the hollow tubular member, the filter member completely surrounding and enclosing the second opening, the filter member defining an enclosed volume and surface area and wherein a ratio between the cross-sectional area of the second opening and the surface area of the filter member is from about 1:5 to about 1:200.

2. A perfusion apparatus as defined in claim 1, wherein the filter member comprises a porous mesh, the mesh having an average pore size of greater than about 60 microns.

3. A perfusion apparatus as defined in claim 1, wherein the filter member comprises a porous mesh, the mesh having an average pore size of greater than about 80 microns.

4. A perfusion apparatus as defined in claim 1, wherein the filter member comprises a porous mesh, the mesh having an average pore size of from about 60 microns to about 150 microns.

5. A perfusion apparatus as defined in claim 1, wherein the ratio between the cross-sectional area of the second opening and the surface area of the filter member is from about 1:15 to about 1:100.

6. A perfusion apparatus as defined in claim 1, wherein the hollow tubular member is made from stainless steel.

7. A perfusion apparatus as defined in claim 1, wherein the perfusion apparatus comprises a single-use perfusion apparatus.

8. A perfusion apparatus as defined in claim 1, wherein the hollow tubular member is made from a thermoplastic polymer and wherein the filter member comprises a polyamide mesh.

9. A perfusion apparatus as defined in claim 1, wherein the hollow tubular member includes a first straight section, a second straight section, and an angled section positioned between the first straight section and the second straight section, the angled section for locating the second opening and filter member at a location in a bioreactor without contacting a rotating impeller.

10. A perfusion apparatus as defined in claim 1, wherein the hollow tubular member includes an angular member located adjacent to the second end, the hollow tubular member including a straight section that transitions into the angular member, the angular member being at an angle to the straight section of from about 50° to about 90°.

11. A perfusion apparatus as defined in claim 1, wherein the hollow tubular member and the filter member are movably enclosed in a collapsible bellows, wherein the collapsible bellows includes a sterile connection port on one end for connecting to a matching sterile connection port of the bioreactor.

12. A perfusion apparatus comprising:
a hollow tubular member for perfusing fluid from a bioreactor, the hollow tubular member having a first end defining a first opening and a second end defining a second opening, the second opening having a cross-sectional area; and
a filter member located and attached to the second end of the hollow tubular member, the filter member completely surrounding and enclosing the second opening, the filter member comprising a porous mesh, the porous mesh having an average pore size of about 60 microns or greater to about 150 microns or less.

13. A perfusion apparatus as defined in claim 12, wherein the filter member defines an enclosed volume and surface area and wherein a ratio between the cross-sectional area of the second opening and the surface area of the filter member is from about 1:5 to about 1:200, such as from about 1:15 to about 1:100.

14. A perfusion apparatus as defined in claim 12, wherein the mesh of the filter member has a uniform pore size.

15. A perfusion apparatus as defined in claim 12, wherein the mesh comprises a stainless steel screen.

16. A perfusion apparatus as defined in claim 12, wherein the hollow tubular member includes a first straight section, a second straight section, and an angled section positioned between the first straight section and the second straight section, the angled section for locating the second opening and filter member at a location in a bioreactor without contacting a rotating impeller.

17. A perfusion apparatus as defined in claim 12, wherein the hollow tubular member includes an angular member located adjacent to the second end, the hollow tubular member including a straight section that transition into the angular member, the angular member being at an angle to the straight section of from about 50° to about 90°.

18. A perfusion apparatus as defined in claim 12, wherein the hollow tubular member and the filter member are movably enclosed in a collapsible bellows, wherein the collapsible bellows includes a sterile connection port on one end for connecting to a matching sterile connection port of the bioreactor.

19. A perfusion apparatus as defined in claim 12, wherein the filter member includes a mesh patch on a side or bottom wall of a bioreactor, the filter member further including a cone connecting the mesh patch to the hollow tubular member.

20. A bioreactor system including a bioreactor having a bioreactor volume, the bioreactor containing microcarriers for fostering cell growth thereon, the bioreactor having a top and defining a port and wherein the bioreactor system further comprises the perfusion apparatus as defined in claim 12, the perfusion apparatus being received within the port.

21. A bioreactor system including a bioreactor having a bioreactor volume, the bioreactor containing microcarriers for fostering cell growth thereon, the bioreactor system further including a perfusion apparatus as defined in claim 19, wherein the bioreactor is in communication with the perfusion apparatus.

22. A method for culturing cell growth comprising:
inoculating biological cells into a bioreactor, the bioreactor containing a fluid medium for cell growth, the biological cells being attached to microcarriers contained within the bioreactor;
perfusing the fluid medium contained in the bioreactor by inserting into the bioreactor a perfusion apparatus, the perfusion apparatus including a hollow tubular member having a first end defining a first opening and a second and opposite end defining a second opening, the perfusion apparatus further including a filter member located and attached to the second end of the hollow tubular member, the filter member completely surrounding and enclosing the second opening and wherein the fluid medium is withdrawn from the bioreactor through the perfusion apparatus, the filter member preventing the microcarriers from being withdrawn from the bioreactor; and
replenishing the fluid medium within the bioreactor in order to promote cell growth.

23. A method for culturing cell growth comprising:
inoculating biological cells into a bioreactor, the bioreactor containing a fluid medium for cell growth, the biological cells being attached to microcarriers contained within the bioreactor;
perfusing the fluid medium contained in the bioreactor placing the bioreactor in communication with a perfusion apparatus, the perfusion apparatus including a hollow tubular member having a first end defining a first opening and a second and opposite end defining a second opening, the perfusion apparatus further including a filter member formed as a mesh patch in a wall of the bioreactor, the perfusion apparatus further including a flexible cone connecting from the mesh patch to the second opening of the hollow tubular member, and wherein the fluid medium is withdrawn from the bioreactor through the perfusion apparatus, the filter member preventing the microcarriers from being withdrawn from the bioreactor; and
replenishing the fluid medium within the bioreactor in order to promote cell growth.
